(19) United States
(12) Patent Application Publication
(10) Pub. No.: US 2025/0072975 A1
(43) Pub. Date: Mar. 6, 2025

(54) REAL-TIME TRACKING AND MONITORING OF ARRAYS FOR VERIFICATION

(71) Applicant: Medos International Sarl, Le Locle (CH)

(72) Inventor: Scott Cazier, Boston, MA (US)

(73) Assignee: Medos International Sarl, Le Locle (CH)

(21) Appl. No.: 18/241,597

(22) Filed: Sep. 1, 2023

(51) Int. Cl. A61B 34/20; A61B 90/00

(52) U.S. Cl. CPC A61B 34/20; A61B 90/39; A61B 2034/2057; A61B 2090/3916; A61B 2090/3937

(57) ABSTRACT

Systems, methods, and apparatus are described herein for real-time tracking and monitoring of arrays and bones during a surgical procedure. For example, a tracking array (e.g., a surveillance marker) may be affixed to an implant to be inserted in a patient. The implant may be, for example, a pedicle screw or a cage. Affixing the tracking array to the implant may allow the surgeon to monitor the patient array location without an additional incision and/or a second imaging (e.g., additional patient radiation exposure).

### TECHNICAL FIELD

Various exemplary embodiments disclosed herein relate generally to real-time tracking and monitoring of arrays for verification.

### BACKGROUND

Navigated and robotic surgery may require a patient reference frame to locate a patient in space. Some embodiments may use a surveillance marker that requires its own separate incision, and which can be used as a reference to determine the distance between the patient reference frame and the surveillance marker.

### SUMMARY

A summary of various exemplary embodiments is presented below. Some simplifications and omissions may be made in the following summary, which is intended to highlight and introduce some aspects of the various exemplary embodiments, but not to limit the scope of the disclosure. Detailed descriptions of an exemplary embodiment adequate to allow those of ordinary skill in the art to make and use the inventive concepts will follow in later sections.

Systems, methods, and apparatus are described herein for real-time tracking and monitoring of arrays during a surgical procedure. For example, a tracking array (e.g., a surveillance marker) may be affixed to an implant to be inserted in a patient. The implant may be, for example, a pedicle screw or a cage. Affixing the tracking array to the implant may allow the surgeon to monitor the patient array location without an additional incision and/or a second imaging (e.g., additional patient radiation exposure).

For example, various embodiments may include a system (e.g., a computer-aided surgery (CAS) system) that comprises a camera and a processor configured to perform certain steps. The camera may be a stereoscopic camera. The processor may determine the location of a first marker attached to the spine of a patient. For example, the first marker may be attached to the patient's spinous process or iliac crest. The processor may determine the location of the first marker based on one or more images of the patient's anatomy. For example, the images may be CT and/or MRI images. The processor may provide navigation information for a first implant based on the location of the first marker (e.g., without monitoring a surveillance marker). The processor may determine the location of surveillance marker that has been attached to the implant. The processor may determine a reference locational relationship (e.g., a distance, 3D models of points in space, etc.) between the first marker and the surveillance marker.

The processor may monitor locations of the first marker and the surveillance marker, and may compute a second locational relationship between the first marker and the surveillance marker. The processor may provide instructions to a user to perform a surgical procedure using the first marker and the surveillance marker. The processor may perform one or more actions if the second locational relationship has deviated from the reference locational relationship by more than a threshold. For example, the processor may stop providing instructions for the surgical procedure, alert the user, prompt the user to modify a location of the first marker, and/or prompt the user to modify a location of the surveillance marker. The processor may provide navigation information for a second implant based on the location of the first marker and the surveillance marker. The processor may determine that the surveillance marker has been attached to the second implant, and may update the reference locational relationship between the first marker and the surveillance marker.

### BRIEF DESCRIPTION OF THE DRAWINGS

In order to better understand various exemplary embodiments, reference is made to the accompanying drawings:

FIG. 1 is a diagram that illustrates a system for real-time tracking and monitoring of arrays;

FIG. 2 is a diagram that illustrates a first marker and a surveillance marker attached to an implant;

FIG. 3 is a flowchart of an example procedure using a surveillance marker attached to an implant during a surgical procedure; and

FIG. 4 a flowchart of another example procedure using a surveillance marker attached to an implant during a surgical procedure.

FIG. 5 is a block diagram illustrating an example of a device that may be used in a CAS system.

To facilitate understanding, identical reference numerals have been used to designate elements having substantially the same or similar structure and/or substantially the same or similar function.

### DETAILED DESCRIPTION

The description and drawings illustrate the principles of the disclosure. It will thus be appreciated that those skilled in the art will be able to devise various arrangements that, although not explicitly described or shown herein, embody the principles of the disclosure and are included within its scope. Furthermore, all examples recited herein are principally intended expressly to be for pedagogical purposes to aid the reader in understanding the principles of the disclosure and the concepts contributed by the inventor(s) to furthering the art and are to be construed as being without limitation to such specifically recited examples and conditions. Additionally, the term, “or,” as used herein, refers to a non-exclusive or (i.e., and/or), unless otherwise indicated (e.g., “or else” or “or in the alternative”). Also, the various embodiments described herein are not necessarily mutually exclusive, as some embodiments can be combined with one or more other embodiments to form new embodiments.

One or more of the embodiments disclosed herein may be used with computer-aided surgery (CAS). Before computer-aided surgery takes place, the CAS system learns the locations and relationships of various elements like the patient (e.g., based on images of the patient which might be obtained by a fluoroscopy, x-ray, CT, MRI, etc.) and medical instruments (e.g., scalpel, saw, drill, bone screw, implant, robot, etc.).

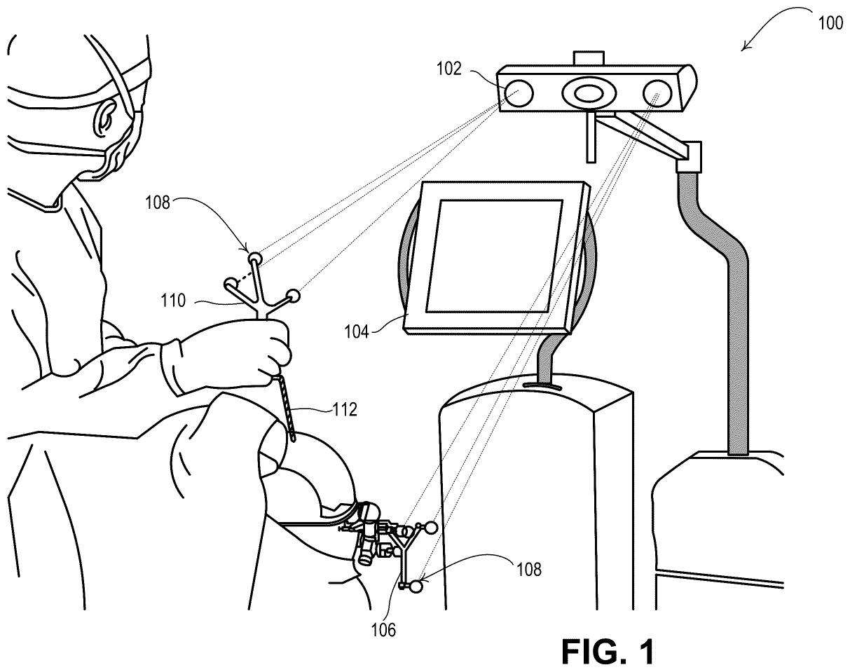

FIG. 1 is a diagram that illustrates a system 100 (e.g., a computer-aided surgery (CAS) system) for real-time tracking and monitoring of arrays. The system 100 may include, for example, a camera 102 (e.g., a stereoscopic and/or spatial camera) and/or a processor (not shown) that may be communicatively coupled to the camera 102. The processor may be part of a computer, and may be configured to perform one or more steps as part of any of the procedures described herein. The processor may further be communicatively coupled to a display (e.g., the display 104 shown in FIG. 1), that may provide a surgeon with instructions for performing a surgical procedure, and/or a memory (not shown).

To enable the CAS to locate the patient, the patient typically has a navigation array attached somewhere on their body, often attached to a bone for stability. For example, as shown in FIG. 1, a navigation array 106 (e.g., which may be referred to herein as a “first marker,” a “first navigational array,” and/or a “reference marker”) may be used to create a patient reference frame to locate the patient in space. These navigation arrays may be monitored by a location device or system such as a spatial camera, one of which is commercially available from Northern Digital Inc. For example, as shown in FIG. 1, the camera 102 may be a stereoscopic camera (e.g., a spatial camera) that is able to determine the location of one or more markers (e.g., including the navigation array 106) in 3-dimensional (3D) space. Spatial cameras typically use an internal coordinate system that is defined by the camera, not by the location of the patient (the spatial camera can be placed in various locations relative to the patient).

The navigation array 106 may be an array of reflective spheres 108 that reflect light back to the spatial camera 102. For example, the spatial camera 102 or other light source might emit infrared (IR) light, and the spatial camera 102 may sense the IR light reflected back from the spheres, and thereby enabling the spatial camera 102 to spatially locate the spheres 108. Alternatively, the navigation array 106 can include LEDs (e.g., or other point light sources) that emit light that will be sensed by the spatial camera 102, for example, without the need for light to be reflected. Further, in some examples, the system 100 might use electromagnetic devices that emit signals that can be used to determine their spatial location by a receiver, or other known systems for navigation of devices, for example, instead of navigation arrays 106 and the spatial camera 102.

Many surgeries use imaging devices (e.g., fluoroscope, x-ray, CT, MRI) that take images of the patient which can be helpful to the surgeon during surgery. Fiducials, such as radiopaque markers, can be attached to the patient before the imaging occurs. These fiducials make relatively well-defined landmarks in the image which can be used later to transform between the patient coordinate system and the camera coordinate system. The imaging devices typically have their own internal coordinate system that is defined by the imaging device itself and has no fixed relation to the coordinate system of the spatial camera 102 (e.g., the spatial camera 102 is typically placed in various locations relative to the imaging device).

Navigation arrays can also be attached to surgical instruments so that the CAS system can track the spatial location of the instrument. For example, as shown in FIG. 1, a navigational array 110 may be attached to a surgical instrument 112 used by a surgeon during a surgical procedure. The spatial camera 102 tracks the location of the navigation array 110, and thus the surgical instrument 112 in the coordinate system of the spatial camera 102. But it is only part of the picture for the spatial camera 102 to know the location of the surgical instrument 112 in the camera coordinate system. It is helpful for the CAS system 100 to be able to know where the instrument 112 is relative to the patient. This happens via a process known as registration described in more detail below. The surgical instrument 112, which may be a pointer device, may be touched to one or more of the patient's spinal processes, and a reading may be taken such that the locations of the spinal processes are determined using the navigational array 110 attached to the surgical instrument 112.

To accomplish this, various processes are used in setting up the CAS system 100 before a surgery. One process is used to allow the CAS system 100 to harmonize between the spatial camera coordinate system, the patient coordinate system, and the image device coordinate system—this process is typically called registration. In registration, the CAS system 100 determines the relationship between the various coordinate systems. That is, if the CAS system 100 knows the spatial relationship between navigation array 106 connected to the patient (e.g., which is monitored by the spatial camera 102) and the fiducials connected to the patient (e.g., which show up in the images created by the imaging device), the CAS system 100 can relate that information mathematically/spatially so that the image of the patient can be appropriately aligned with or overlaid onto the patient in 3D space.

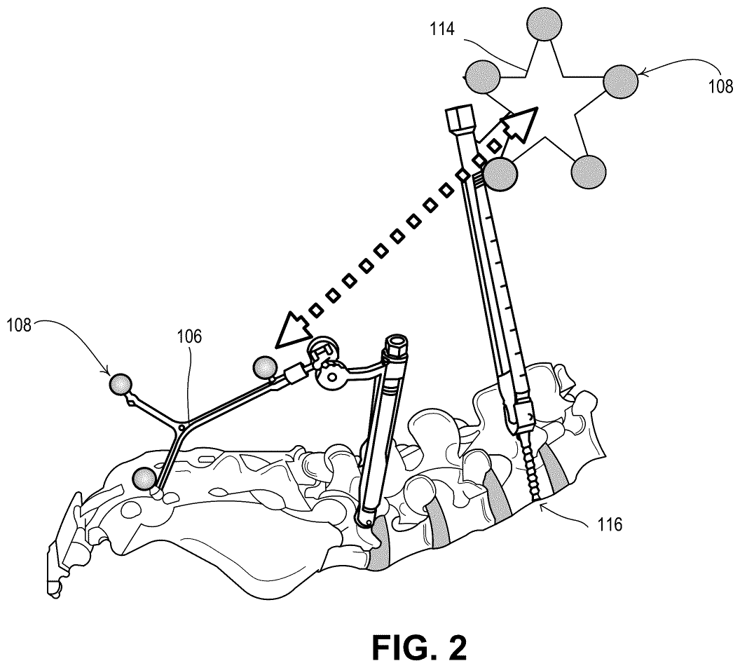

A navigational array that is attached to a patient (e.g., such as the navigational array 106) may be susceptible to unintended motion (e.g., bumps, vibration, sliding on the pins, etc.). For example, as shown in FIG. 2, the navigational array 106 may be hinged to allow some flexibility in its angulation relative to the patient; however, this flexibility may mean that the position and/or location of the navigational array 106 can be moved accidentally even after it is put into place. Therefore, in some embodiments, a second navigational array 114 may be attached to the patient. The second navigational array 114 may be referred to as a “surveillance marker” and/or a “second array.” FIG. 2 is a diagram that illustrates the navigational array 106 and a surveillance marker 114 attached to an implant 116. The second navigational array 114 may be used as a reference for the location of the navigational array 106. For example, as with the navigational array 106, the surveillance marker 114 may be monitored by the spatial camera 102. Although the surveillance marker 114 is shown in FIG. 2 as having five reflective spheres 108, other numbers are possible. For example, the surveillance marker 114 may have three or four reflective spheres 108. The reflective spheres 108 on the surveillance marker 114 may be in an asymmetric configuration.

The spatial camera 102 (e.g., and/or the processor) may determine a location of the surveillance marker 114 relative to the navigational array 106 attached to the patient, and/or may determine a locational relationship (e.g., a reference distance) between the navigational array 106 and the surveillance marker 114. If, during the surgical procedure, the locational relationship between the navigational array 106 and the surveillance marker 114 changes (e.g., increases or decreases) by more than a threshold value, the camera 102 and/or the processor may determine that the location of the navigational array 106 has been changed, at which point the spatial location of the navigational array 106 determined by the camera 102 and/or the processor may no longer be valid. The processor may then perform one or more actions, including but not limited to alerting the surgeon via the display 104 and/or via another form of alert (e.g., an audio and/or visual alert).

In order to ensure that the surveillance marker 114 is useful for monitoring the location of the navigational array 106 attached to the patient, the location of the surveillance marker 114 may be fixed for a given period of time. One way to ensure that the location of the surveillance marker 114 is fixed is to create another incision on the patient located away from the surgical site, and to attach the surveillance marker 114 there. However, doing so often requires another incision in addition to those necessary for the surgery.

Therefore, a system may be used whereby the surveillance marker 114 is attached to an implant 116 that is implanted into the patient during the surgical procedure. As described above, processor (e.g., in conjunction with the spatial camera 102) may use the surveillance marker 114 may be used to determine a reference locational relationship relative to a navigational array 106 that is attached to the patient (e.g., the patient's spine or the patient's iliac crest). Attaching the surveillance marker 114 to the implant 116 may reduce the number of incisions necessary, since the surveillance marker 114 will not require a separate incision to be attached to the patient.

For example, as shown in FIG. 2, the navigational array 106 (e.g., which may be referred to as a “first navigational array,” a “first marker,” and/or “a reference marker”) may be attached to the patient's spine. As described herein, the navigational array 106 and/or the surveillance marker 114 may be or include an array of reflective spheres 108 that reflect light back to the spatial camera 102, LEDs (e.g., or other point light sources) that emit light that will be sensed by the spatial camera 102 (e.g., no reflection is required), and/or electromagnetic devices that emit signals that can be used to determine their spatial location by a receiver, or other known systems for navigation of devices.

Using the surveillance marker 114 may allow the system to compare the position or location of the surveillance marker 114 to the position or location of the navigational array 106. A surgeon may attach the surveillance marker 114 to an implant 116 that is inserted into the patient's spine as part of a surgical procedure. Alternatively, the surveillance marker 114 may be attached to another type of implant, including but not limited to a pedicle screw, a cage, a plate for trauma, a prosthesis (e.g., shoulder, knee, etc.). As described above, the spatial camera 102 may monitor the location of the surveillance marker 114 with reference to the navigational array 106 (e.g., a reference locational relationship).

One or more methods for real-time tracking and monitoring of arrays may be disclosed herein. For example, any of the methods disclosed herein, and/or portions thereof, may be performed by one or more portions of the system disclosed in FIG. 1, and may use one or more navigational arrays or markers (e.g., the navigational array 106 and/or the surveillance marker 114, each of which is described in further detail with reference to FIGS. 1 and 2).

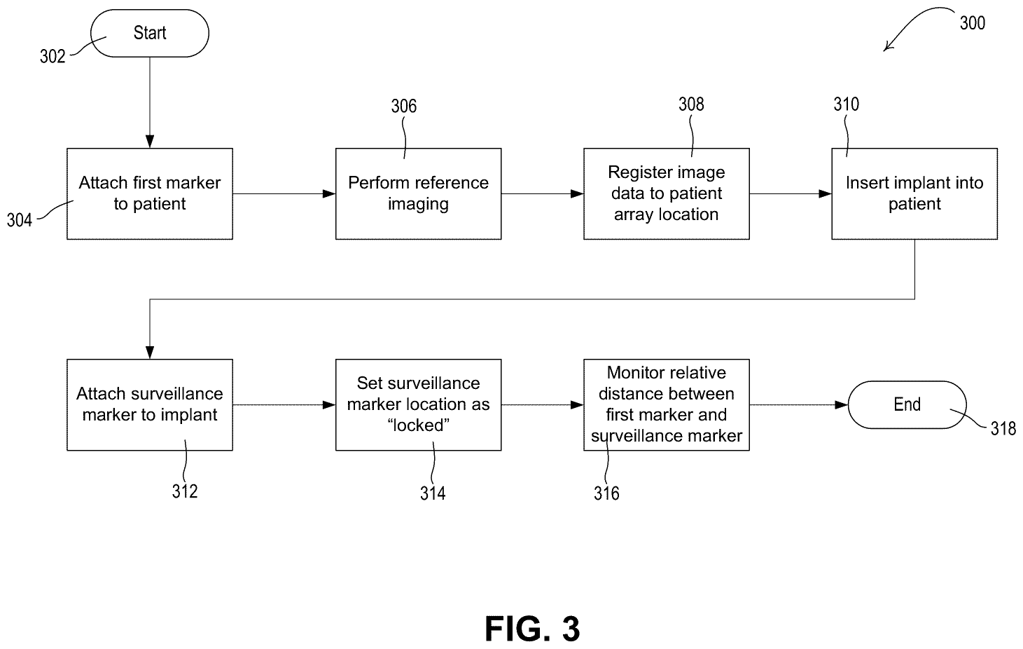

FIG. 3 is a flowchart of an example procedure 300 using a surveillance marker attached to an implant during a surgical procedure. One or more of the steps of the procedure 300 may be performed in conjunction with a CAS system, which may include a processor, such as the processor described with reference to FIG. 1, and/or a camera, such as the stereoscopic camera 102 described with reference to FIG. 1. One or more of the steps of the procedure 300 may be performed by a medical professional (e.g., a surgeon).

The procedure 300 may begin at 302. At 304, the surgeon may attach a first navigational array to a patient. The first navigational array may be referred to as a “first array” and/or as a “first marker” herein. The first marker may be as described with reference to FIG. 1. For example, the first marker may be or include an array of reflective spheres that reflect light back to the stereoscopic camera, LEDs (e.g., or other point light sources) that emit light that will be sensed by the spatial camera (e.g., no reflection is required), and/or electromagnetic devices that emit signals that can be used to determine their spatial location by a receiver, or other known systems for navigation of devices. The surgeon may attach the first marker to a spine of a patient (e.g., a spinous process or iliac crest) during a surgical procedure. For example, the location of the first marker may be or include one or more of a navigation array configured and adapted to connect to a spinous process of the patient and a navigation array configured and adapted to connect to an iliac crest of the patient.

At 306, reference imaging may be performed. For example, as described with reference to FIG. 1, the reference imaging may use imaging devices (e.g., fluoroscope, x-ray, CT, MRI) that take images of the patient which can be helpful to the surgeon during surgery. The reference imaging may use one or more fiducials, such as radiopaque markers, that can be attached to the patient before the imaging occurs. Although the reference imaging is described as being performed after the first marker is attached to the patient (e.g., during the surgical procedure), the reference imaging may alternatively be performed prior to attaching the first marker to the patient (e.g., prior to the surgical procedure). At 308, the reference image data may be registered to the patient array location. The reference imaging may be combined with images taken by a stereoscopic camera to determine a location of the first marker with reference to the patient's anatomy. For example, fiducials used in the reference imaging may be used as relatively well-defined landmarks in the image which can be used to transform between the patient coordinate system and the camera coordinate system. By performing this registration, the system may acquire the location of the reference array relative to the patient, giving a fixed location for the first marker.

At 310, the surgeon may insert an implant into a patient. For example, as described herein, the implant may be a pedicle screw, a cage, a plate for trauma, a prosthesis (e.g., shoulder, knee, etc.), and/or the like. The surgeon may insert the implant into the patient's spine as part of the surgical procedure. After the implant is inserted into the patient at 310, a second navigational array may be attached to the implant. The second navigational array may be referred to as a “second array” and/or as a “surveillance marker” herein. The second navigational array may be as described with reference to FIG. 2. For example, the second navigational array may be or include an array of reflective spheres that reflect light back to the spatial camera, LEDs (e.g., or other point light sources) that emit light that will be sensed by the spatial camera (e.g., no reflection is required), and/or electromagnetic devices that emit signals that can be used to determine their spatial location by a receiver, or other known systems for navigation of devices. The second navigational array may be attached to the implant via a fastening mechanism, such as a screwing mechanism, a clasping mechanism, pins, and/or the like, and preferably a secure fastening mechanism that is not likely to be disturbed by a bump during the surgical procedure.

At 314, the surgeon and/or the CAS system may set the location of the surveillance marker as “locked.” For example, setting the location of the surveillance marker may include fixing the surveillance marker to the implant such that there is no further movement of the surveillance marker relative to the implant or to the patient (e.g., the surveillance marker is tightly threaded to the implant with male/female thread coupling, for example), and/or providing an indication that the location of the surveillance marker is fixed (this could done via the surgeon indicating via a GUI that the array is in place and secured to the implant and that the implant has been implanted). The processor in conjunction with the stereoscopic camera may then determine and/or acquire the location of the surveillance marker with reference to the first marker. For example, the processor in conjunction with the stereoscopic camera may determine a reference locational relationship (e.g., a relative distance) between the first marker and the surveillance marker once the location of the surveillance marker is set as locked. Performing this step may enable the surgeon to ensure that the relative distance between the first marker and the surveillance marker is correctly measured, as premature calculations while the surveillance marker is still being moved by the surgeon may not be valid. Although the locational relationship between the first marker and the surveillance marker is discussed herein as being a relative distance, in other examples, the locational relationship is any type of measurement that relates the location of the first marker and the surveillance marker.

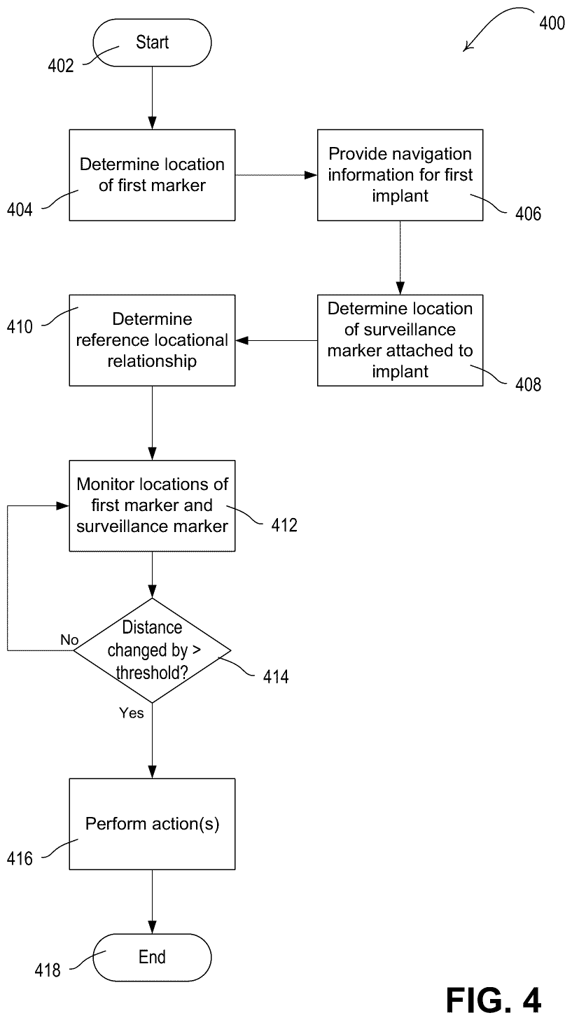

After the location of the surveillance marker is locked at 314, the spatial camera (e.g., in conjunction with the processor) may monitor relative distance between the first marker and the surveillance marker may be monitored at 316. For example, the relative distance may be monitored by the stereoscopic camera (e.g., the camera 102). The relative distance may change over time due to a number of factors, including motion of the implant from its originally implanted position, and/or unintended motion of the first marker with respect to the patient's body (e.g., bumps, vibration, sliding on the pins, etc.). If the relative distance changes (e.g., by more than a threshold value), the surgeon and/or the processor may perform one or more actions. Further detail regarding the actions to be performed can be found with reference to FIG. 4. The monitoring of the relative distance may continue throughout the surgical procedure, and may end when the surgeon sets the location of the surveillance marker as unlocked, and/or when the surgeon indicates that the surgical procedure has been completed. The procedure 300 may end at 318.

As noted above, while one or more steps of the procedure 300 are described with reference to actions performed by a surgeon during a surgical procedure, these steps may be performed in conjunction with a CAS system. For example, the CAS system may include a processor, a memory, a display, and/or a stereoscopic camera. The CAS system may be used to provide information and/or instructions to the surgeon before and during the surgical procedure. FIG. 4 provides further details regarding the actions taken by the CAS system during the surgical procedure.

FIG. 4 a flowchart of another example procedure 400 using a surveillance marker attached to an implant during a surgical procedure. The procedure 400 may be performed by a CAS system (e.g., the CAS system 100), which may include a processor, such as the processor described with reference to FIG. 1, and/or a stereoscopic camera, such as the stereoscopic camera 102 described with reference to FIG. 1. The procedure 400 may be stored in a memory as computer-readable or machine-readable instructions that may be executed by the processor of one or more devices for executing the procedure 400. The memory may be communicatively coupled to the processor. Though the procedure 400 may be described herein as being performed by a single device, such as a processor, the procedure 400, or portions thereof, may be performed by another device or distributed across multiple devices, such as a wired/wireless processor and/or one or more other devices.

The procedure 400 may begin at 402. At 404, the processor may determine the location of a first navigational array (e.g., which may be referred to as a “first marker” and/or as a “first array”). For example, the processor may determine the location of the first marker relative to a patient reference frame using images from an imaging device (e.g., MRI, CT, x-ray, fluoroscopy, etc.). The location of the first marker may be determined by the processor in conjunction with a stereoscopic camera to which the processor is communicatively coupled. The first marker may be or include an array of reflective spheres that reflect light back to the stereoscopic camera, LEDs (e.g., or other point light sources) that emit light that will be sensed by the spatial camera (e.g., no reflection is required), and/or electromagnetic devices that emit signals that can be used to determine their spatial location by a receiver, or other known systems for navigation of devices. The location of the first marker relative to the patient reference frame may be stored in a memory communicatively coupled to the processor. The first marker may be located on (e.g., attached to) a spine of the patient (e.g., a spinous process or an iliac crest).

At 406, the processor may provide navigation information for a first implant. For example, the processor may provide the navigation information via a display that is communicatively coupled to the processor. The processor may provide the navigation information to a surgeon as part of a surgical procedure on the patient. The processor may determine the navigation information based on the determined location of the first marker, and may provide the determined navigation information.

At 408, the processor may determine a location of a second navigational array (e.g., which may be referred to as a “second array” and/or as a “surveillance marker”). The location of the first marker may be determined by the processor in conjunction with the stereoscopic camera. For example, the surveillance marker may be or include a single marker/reflective sphere or an array of reflective spheres that reflect light back to the stereoscopic camera, LEDs (or other point light sources) that emit light that will be sensed by the spatial camera (no reflection is required), and/or electromagnetic devices that emit signals that can be used to determine their spatial location by a receiver, or other known systems for navigation of devices. The surveillance marker may be attached to the first implant. The processor may receive an indication that a location of the surveillance marker is “locked,” and the processor may then determine the location of the surveillance marker.

At 410, the processor may determine a reference locational relationship between the first marker and the surveillance marker. For example, the processor may determine a relative distance between the first marker and the surveillance marker based on images received from the stereoscopic camera. Alternatively, the reference locational relationship may be 3D models of points in space. The reference locational relationship may be stored in the memory and may be measured in any applicable units, and/or may be determined using a unitless system. For example, an initial locational relationship between the first marker and the surveillance marker may be set, and any change in the locational relationship may be measured with reference to the initial value. In an example, the initial locational relationship between the first marker and the surveillance marker may be determined to be approximately 25 centimeters (cm) along a certain vector. The processor may then store the value of 25 centimeters and the vector in memory.

At 412, the processor may monitor the locations of the first marker and the surveillance marker, and/or the locational relationship (e.g., distance along a certain vector) between the two markers. For example, the processor may monitor the locations of the markers during the surgical procedure. The processor may monitor the locations of the markers using images received from the stereoscopic camera. The system may provide navigation information for a second implant, for example via the display, based on the monitoring of the locations of the first marker and optionally the surveillance marker. For example, the system may use the locations of the first marker and optionally the surveillance marker (e.g., and or the locational relationship between the two markers) to provide the navigational information for the second implant. Once the second implant has been inserted into the patient, the processor may determine that the surveillance marker has been detached from the first implant and attached to the second implant, for example based on an indication received from the surgeon. The processor may then determine an updated reference locational relationship (e.g., an updated relative distance) between the first marker and the surveillance marker, and may use the updated reference locational relationship in later steps. Alternatively, the surveillance marker may remain secured to the first implant.

At 414, the processor, in conjunction with the stereoscopic camera, may determine whether the locational relationship between the first marker and the surveillance marker has changed by more than a threshold value compared to the reference locational relationship at 414. The threshold value may be stored in the memory and/or set by the surgeon (e.g., or another user) prior to the surgical procedure. If the processor determines that the locational relationship between the first marker and the surveillance marker has not changed by an amount that is more than the threshold value at 414, the procedure 400 may return to 412, and the processor may continue to monitor the locations of the first marker and the surveillance marker for any further changes. Alternatively, if the processor determines that the locational relationship between the first marker and the surveillance marker has changed by an amount that is more than the threshold value at 414, the processor may perform one or more actions at 416 based on the determination that the locational relationship has changed by an amount that is more than the threshold value. The processor may then perform one or more actions, including but not limited to alerting the surgeon via the display and/or via another form of alert (e.g., an audio and/or visual alert). Additionally or alternatively, the processor (e.g., via the display) may stop providing instructions for the surgical procedure, prompt the surgeon to modify a location of the first marker, and/or prompt the user to modify a location of the surveillance marker. For example, the processor may prompt the surgeon to return the first marker to its previous location, and may provide instructions to return the first marker to the previous location via the display, and to re-run the registration process. The processor may receive an indication from the surgeon that the first marker has been returned to its previous location, and the surgical procedure may continue. The procedure 400 may end at 418.

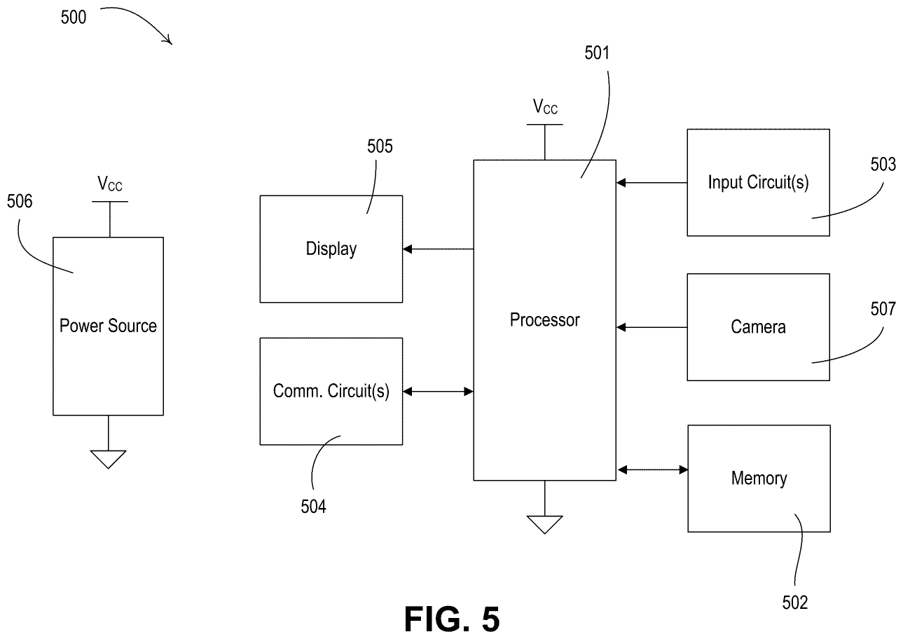

FIG. 5 is a block diagram illustrating an example of a device 500 that may be used in a CAS system. For example, the device 500 may be used in the CAS system 100 described in FIG. 1.

Referring again to FIG. 5, the device 500 may include a processor 501 for controlling the functionality of the device 500. The processor 501 may include one or more general purpose processors, special purpose processors, conventional processors, digital signal processors (DSPs), microprocessors, integrated circuits, a programmable logic device (PLD), application specific integrated circuits (ASICs), and/or the like. The processor 501 may perform any functionality that enables the device 500 to perform as described herein. For example, the processor 501 may perform one or more of the steps of the procedures 300 and/or 400. The processor 501 may include and/or may be the processor described with reference to FIGS. 1-4.

The processor 501 may be communicatively coupled to a memory 502, and may store information in and/or retrieve information from the memory 502. The memory 502 may comprise computer-readable storage media and/or machine-readable storage media that maintains any values or indicators described herein, and/or computer-executable instructions for performing as described herein. For example, the memory 502 may comprise computer-executable instructions or machine-readable instructions that include one or more portions of the procedures described herein. The processor 501 may access the instructions from memory 502 for being executed to cause the processor 501 to operate as described herein, or to operate one or more other devices as described herein. The memory 502 may comprise computer-executable instructions for executing configuration software and/or control software. The computer-executable instructions may be executed to perform one or more procedures described herein.

The memory 502 may include a non-removable memory and/or a removable memory. The non-removable memory may include random-access memory (RAM), read-only memory (ROM), a hard disk, and/or any other type of non-removable memory storage. The removable memory may include a subscriber identity module (SIM) card, a memory stick, a memory card, and/or any other type of removable memory. The memory 502 may be implemented as an external integrated circuit (IC) or as an internal circuit of the processor 501.

The device 500 may include one or more communication circuits 504 that are in communication with the processor 501 for sending and/or receiving information as described herein. The communication circuit 504 may perform wireless and/or wired communications. The communication circuit 504 may be a wired communication circuit capable of communicating on a wired communication link. The wired communication link may include an Ethernet communication link, an RS-485 serial communication link, a 0-10 volt analog link, a pulse-width modulated (PWM) control link, and/or another wired communication link. The communication circuit 504 may be configured to communicate via power lines (e.g., the power lines from which the device 500 receives power) using a power line carrier (PLC) communication technique. The communication circuit 504 may be a wireless communication circuit including one or more RF or infrared (IR) transmitters, receivers, transceivers, and/or other communication circuits capable of performing wireless communications.

Though a single communication circuit 504 is illustrated in FIG. 5, multiple communication circuits may be implemented in the device 500. The device 500 may include a communication circuit configured to communicate via one or more wired and/or wireless communication networks and/or protocols, and at least one other communication circuit configured to communicate via one or more other wired and/or wireless communication networks and/or protocols. For example, a first communication circuit may be configured to communicate via a wired or wireless communication link, while another communication circuit may be capable of communicating on another wired or wireless communication link. The first communication circuit may be configured to communicate via a first wireless communication link (e.g., a wireless network communication link) using a first wireless protocol (e.g., a wireless network communication protocol), and the second communication circuit may be configured to communicate via a second wireless communication link (e.g., a short-range or direct wireless communication link) using a second wireless protocol (e.g., a short-range wireless communication protocol).

The processor 501 may be in communication with one or more input circuits 503 from which inputs may be received. For example, the input circuits 503 may include, but are not limited to, one or more buttons, a touchscreen, a voice-activated input, a foot pedal, an augmented reality (AR) eye gaze, and/or the like.

The processor 501 may be in communication with a display 505. The display 505 may include one or more indicators (e.g., visible indicators, such as LEDs) for providing indications (e.g., feedback). The display 505 may be a visible display for providing information (e.g., feedback) to a user. The processor 501 and/or the display may generate a graphical user interface (GUI) generated via software for being displayed on the device 500 (e.g., on the display 505 of the device 500). For example, the display 505 may be the display 104 described with reference to FIG. 1.

Each of the hardware circuits within the device 500 may be powered by a power source 506. The power source 506 may include a power supply configured to receive power from an alternating-current (AC) power supply or direct-current (DC) power supply, for example. The power source 506 may produce a supply voltage for powering the hardware within the device 500.

The processor 501 may be in communication with a camera 507. For example, the camera 507 may be a stereoscopic (e.g., spatial) camera. The camera 507 may be the spatial camera 102 described with reference to FIG. 1. The spatial camera may be used (e.g., in conjunction with the processor) to determine the location of one or more navigational arrays and/or markers as described herein.

Although the various exemplary embodiments have been described in detail with particular reference to certain exemplary aspects thereof, it should be understood that the disclosure is capable of other embodiments and its details are capable of modifications in various obvious respects. As is readily apparent to those skilled in the art, variations and modifications and combinations of the various embodiments can be affected while remaining within the spirit and scope of the disclosure. Accordingly, the foregoing disclosure, description, and figures are for illustrative purposes only and do not in any way limit the disclosure, which is defined only by the claims.

What is claimed is:

1. A system comprising:
a camera; and
a processor configured to:
determine a location of a first marker attached to a spine of a patient;
provide navigation information for a first implant based on the location of the first marker;
determine a location of surveillance marker that has been attached to the implant;
determine a reference locational relationship between the first marker and the surveillance marker;
monitor a second locational relationship between the first marker and the surveillance marker; and
if the second locational relationship deviates from the reference locational relationship by more than a threshold value, perform one or more actions.

2. The apparatus of claim 1, wherein the location of the first marker comprises one or more of a navigation array configured and adapted to connect to a spinous process of the patient and a navigation array configured and adapted to connect to an iliac crest of the patient.

3. The apparatus of claim 1, wherein the camera is a stereoscopic camera.

4. The apparatus of claim 1, wherein the processor is further configured to provide instructions to a user to perform a surgical procedure using the first marker.

5. The apparatus of claim 4, wherein the processor being configured to perform the one or more actions comprises the processor being configured to one or more of stop providing instructions for the surgical procedure, alert the user, prompt the user to modify a location of the first marker, prompt the user to modify a location of the surveillance marker, and perform re-registration.

6. The apparatus of claim 4, wherein the processor being configured to provide the instructions to the user to perform the surgical procedure using the first marker and the surveillance marker comprises the processor being configured to provide navigation information for a second implant based on the location of the first marker.

7. The apparatus of claim 1, wherein the processor is configured to provide the navigation information for the first implant prior to connection of the surveillance marker to the first implant. 8 The apparatus of claim 1, wherein the reference locational relationship comprises a distance between the first marker and the surveillance marker along a vector.

9. The apparatus of claim 1, wherein the processor is further configured to lock the location of the first marker.

10. The apparatus of claim 1, wherein the processor is further configured to:
determine that a second implant has been inserted into the spine of the patient;
determine that the second surveillance marker has been attached to the second implant; and
determine an updated reference locational relationship between the first marker and the surveillance marker.

11. A method comprising:
determining a location of a first marker attached to a spine of a patient;
providing navigation information for a first implant based on the location of the first marker;
determining a location of surveillance marker that has been attached to the implant;
determining a reference locational relationship between the first marker and the surveillance marker;
monitoring a second locational relationship between the first marker and the surveillance marker; and
if the second locational relationship deviates from the reference locational relationship by more than a threshold value, performing one or more actions.

12. The method of claim 11, wherein the location of the first marker comprises one or more of a navigation array configured and adapted to connect to a spinous process of the patient and a navigation array configured and adapted to connect to an iliac crest of the patient . . .

13. The method of claim 11, wherein the location of the first marker and the location of the surveillance marker are determined based on images received from a stereoscopic camera.

14. The method of claim 11, wherein the reference locational relationship comprises one or more of a distance, a vector, and 3-dimensional (3D) points.

15. The method of claim 11, wherein performing the one or more actions comprises one or more of stopping providing instructions for a surgical procedure, alerting a user, prompting the user to modify a location of the first marker, prompting the user to modify a location of the surveillance marker, and performing re-registration.

16. The method of claim 11, further comprising providing navigation information for a second implant based on the location of the first marker.

17. The method of claim 11, further comprising providing the navigation information for the first implant prior to connection of the surveillance marker to the first implant.

18. The method of claim 11, wherein the reference locational relationship comprises a distance between the first marker and the surveillance marker along a vector.

19. The method of claim 11, further comprising locking the location of the first marker.

20. The method of claim 11, further comprising:
determining that a second implant has been inserted into the spine of the patient;
determining that the second surveillance marker has been attached to the second implant; and
determining an updated reference locational relationship between the first marker and the surveillance marker.
